(19) United States
(12) Patent Application Publication
(10) Pub. No.: US 2008/0011308 A1
(43) Pub. Date: Jan. 17, 2008

(54) Reverse fitting earplug

(76) Inventor: Thomas Walter Fleming, San Diego, CA (US)

Correspondence Address: Maryanne DeAngelo, 1213 Avocet Court, Cardiff by the Sea, CA, 92007, US

(21) Appl. No.: 11/824,951

(22) Filed: Jul. 3, 2007

Related U.S. Application Data
(60) Provisional application No. 60/807,164, filed on Jul. 12, 2006.

(51) Int. Cl. A61F 11/08

(52) U.S. Cl. 128/864

(57) ABSTRACT

A comfortable, less rigid, reverse fitting earplug having at least the equivalent sound blocking surface area contact and seal of conventional push-in earplugs without uncomfortable counter forces on the ear canal from rigidity in the constructive material is disclosed. The reverse fitting earplug comprises concave flanges constructed of soft memory plastic materials, which reverse direction from generally concave forward to generally convex forward upon insertion into the ear canal. Surface contact and seal are maintained by the tendency of the flange material to return to its originally molded concave shape. Pressure forces from rigid structural material are not necessary to hold the reverse fitting earplug in place. The reverse fitting earplug can be comfortably worn for extended periods that cannot be achieved with conventional pressure force reliant designs. When a user removes the reverse fitting earplug the flanges automatically return to their originally molded concave shape.

### CROSS-REFERENCE

The present application claims priority to provisional U.S. Application Ser. No. 60/807,164, entitled “Transforming Earplug,” filed Jul. 12, 2006.

### BACKGROUND

1. Field

The presently disclosed embodiments relate generally to earplugs, and more specifically to a reverse fitting earplug having direction changing flange(s).

2. Background

The need for individual hearing protection in industrial and other occupational and recreational settings is well established. The prior art is replete with hearing protection devices, including earplugs, earmuffs, semi-insert devices, full-head helmets, etc. Such devices are intended to be worn over a user's ear, or are inserted at least partially within the ear canal, to prevent sounds from reaching the inner ear at undesirably high levels.

Earplugs include any of a variety of devices designed to be inserted into the ear canal and are often preferred for providing high attenuation while being discrete and comfortable when worn. Earplugs generally may be categorized as either “roll-down” or “push-in”. Roll-down type earplugs are typically compressible, slow-recovery foam earplugs which must be compressed, or “rolled down”, by the user prior to insertion into the ear canal. Roll-down type earplugs are often composed of a homogenous slow recovery polyvinyl chloride (PVC) or polyurethane (PU) material and include a substantially circular cross-section which is larger than a cross-section of the average ear canal. Roll-down type earplugs are compressed before insertion to reduce the cross-section, allowing insertion into the ear canal. Once inserted, the compressed roll-down earplug expands to occlude the ear canal, blocking the passage of sound into the inner ear.

Conventional push-in type earplugs generally comprise an attenuating portion extending from a rigid or semi-rigid stem portion. The sound attenuating portion is typically formed of a soft conformable material; the rigid or semi-rigid portion may be composed of any material, such as a plastic or a rubber, with sufficient rigidity. Push-in type earplugs are often preferred for their ease of insertion. Unlike roll-down type earplugs, push-in plugs do not require compression prior to insertion. The user simply grasps the rigid or semi-rigid portion and inserts the attenuating portion into the ear canal. Here, the rigid or semi-rigid portion is utilized to push the sound attenuating portion into a sealing position within the ear canal. Upon insertion, the sound attenuating portion conforms to the contours of the ear canal, occluding the ear canal to inhibit the passage of sound. Thus, push-in type earplugs provide for a more convenient insertion process than roll-down type earplugs. Additionally, push-in earplugs are often preferred for their hygienic properties over roll-down plugs because push-in earplugs only require handling of the rigid or semi-rigid stem portion during insertion, minimizing the transfer of substances (e.g., bacteria, dirt, oil, etc.) from the fingers to the attenuating portion, reducing the likelihood that such substances are exposed to the ear canal. For these reasons, roll down type earplugs are typically single use disposable products, while push in type earplugs are reusable, long lasting and economical.

Roll-down and push-in type earplugs are routinely tested for their ability to block sound in a human ear canal. This ability to block sound, or attenuate, is measured in accordance with the established testing procedures such as that set forth in the American National Standards Institute's, “Method for the Measurement of Real-Ear Protection of Hearing Protectors and Physical Attenuation of Earmuffs”, ANSI S3.19-1974. In this test, Real-ear Attenuation at Threshold (REAT) testing is conducted in a laboratory test chamber, which is a semi-reverberant, double-walled, structurally isolated room using third-octave bands of noise as test signals. A human subject responds to the test signals at her/his threshold, i.e. as soon as the subject can detect the signals, in both Open (nothing in or around the ears) and Occluded (hearing protector in the ears) conditions. The difference in sound pressure level (SPL) between the two conditions is the attenuation afforded by the Hearing Protector. This difference is recorded in decibels of attenuation provided by the hearing protector at a given frequency. Data obtained from this test method is then used to calculate a noise reduction rating (NRR), which provides a single number then used to label the product. There is a desire to develop products having higher NRR values without sacrificing comfort, hygiene or ease of insertion.

Roll-down type earplugs often exhibit a higher NRR than comparable push-in type earplugs. However, this higher NRR is dependent upon a proper insertion of the earplug into the ear canal. As mentioned above, insertion of roll-down type earplugs can be more complicated and perhaps more time consuming than insertion of push-in type earplugs. Additionally, due to the foam material construction, roll-down type earplugs often have shorter usage lifetime than push-in earplugs. Push-in plugs, on the other hand, are easily insertable and have a longer usage lifetime but are often associated with lower NNRs than comparable roll-down type earplugs.

Attempts have been made to increase the comfort and NRR provided by push-in type earplugs. These attempts have focused on varying the design or construction of push-in earplugs to attain a better fit or seal within the ear canal. However, these conventional push in constructions have resulted in only moderately increased NRRs at the sacrifice of comfort to the user. Prior design of conventional push-in type earplugs is known to be a tradeoff between comfort and rigidity, which provides a higher NRR rating. Sufficient sound blocking surface area contact and sealing have been created by uncomfortable counter forces on the ear canal from rigidity in the constructive material (i.e the harder the material, the better the NRR rating, and also the more uncomfortable for the user). Existing reusable earplugs rely upon forward inserting rigid convex shaped flanges to provide ease of insertion, noise blocking, and ear canal sealing, which create uncomfortable products for end user wear in work environments where employees are required to wear hearing protection all day long.

Thus, there is a need in the art for a more comfortable, less rigid push-in earplug having at least the equivalent sound blocking surface area contact and seal of conventional earplugs without uncomfortable counter forces on the ear canal from rigidity in the constructive material.

### SUMMARY

Embodiments disclosed herein address the above-stated needs by providing a push-in type earplug having a comfortable seal inside the ear canal created by concave flanges constructed of soft materials, which reverse direction upon insertion into the ear canal. In one aspect, an earplug having at least one direction changing flange is disclosed. In another aspect, an earplug comprising a stem extending in frontward and rearward directions along a stem axis and at least one direction reversing flange extending from the stem is detailed.

### BRIEF DESCRIPTION OF THE DRAWINGS

FIG. 1 is an exemplary partial cross section half view and a partial elevation half view;

FIG. 2 is an alternate exemplary partial cross section half view and partial elevation half view;

FIG. 3 is another alternate exemplary partial cross section half view and partial elevation half view;

FIG. 4A is an exemplary view prior to insertion;

FIG. 4B is an exemplary view during insertion; and

FIG. 5 is an exemplary view after insertion.

### DETAILED DESCRIPTION

The word “exemplary” is used exclusively herein to mean “serving as an example, instance, or illustration.” Any embodiment described herein as “exemplary” is not necessarily to be construed as preferred or advantageous over other embodiments.

The term “material” is used herein to mean any current or future material capable of producing a reversible flange. “Material” may comprise a plastic having any polymer backbone such as polyvinyl chloride, polyethylene, high density polyethylene, low density polyethylene, polyethylene terephthalate, polymethyl methacrylate, polypropylene, polystyrene, naglene, or other acrylics, silicones, polyurethanes, and the like. “Material” may comprise any type of moldable plastic, injection moldable plastic, foam, moldable foam, rubber, saturated rubber, unsaturated rubber, or any elastomer, thermoplastic, thermoplastic elastomer (TPE), thermoplastic rubber, styrene block copolymer elastomer, elastomeric plastic, thermoset, thermoplastic vulcanizates, copolymer or any biodegradable plastic.

The term “elastomer” is used herein to mean any type of elastomer such as Thermoplastic Elastomers (TPE), for example Hytrel®, etc., Thermoplastic Vulcanizates (TPV), for example Santoprene® TPV, Polyurethane rubber, Resilin, Elastin, Polysulfide Rubber, other amorphous polymer, or any biphasic material composed by a plastic phase and a rubbery phase.

All push-in ear plug designs to date utilize tapered flanges having a geometry that is “arrowhead” shaped, or non-reversibly convex. These traditionally designed flanges have prevailed historically due to ease of insertion rather than comfort or wearability. The following figures illustrate a novel and innovative reverse fitting design that counters contemporary thinking by utilizing concave flanges that, when inserted, reverse direction to create a highly comfortable air tight seal. Such a design requires a unique understanding of soft flexible high-tech materials and how they can be used to create maximum surface area contact and seal while maintaining ease of insertion and long wearing comfort. Reversible concave flange(s) are molded from material having softness, flexibility and memory. As the earplug is inserted into the ear canal, the flanges reverse direction from concave to convex, creating a seal based on the material's memory of its original molded shape rather than the material rigidity of all previous ear plug designs. Rather than reliance upon material shaped for entrance into the ear canal, which then depends upon a rigid structure for applying force to make sealing contact against the wall of the ear canal, the memory property of injection molded plastics of low durometer materials allows the concave molded flanges to reverse their shape on contact with the ear canal. This property of injection molded plastics creates a new feature for sealing between the reversible flanges and the ear canal interior walls allowing for softer and more comfortable earplug geometry that provides superior fit and end user comfort.

Additional features and benefits on the present reverse fitting earplug design include a large variety of variations possible for reversible flange geometry. The flanges may be hemispherical, elliptical, straight lined, or any combinations thereof. The number of reversible flanges may vary from at least one to an exemplary four, or more. The thickness of the reversible flanges may be uniform or varied. For example, the front flanges may be thinner than those at the rear of the earplug or vice versa. The diameter of the flanges may similarly vary. The following disclosed embodiments readily lend themselves to any permutation of the number, thickness and diameter of reversible flanges. The reverse fitting earplug is an ideal ear interface for electronic ear speakers, hearing aids, and speech therapy or other electronic devices, as well as any consumer communication device such as a cell phone, radio, ipod and the like.

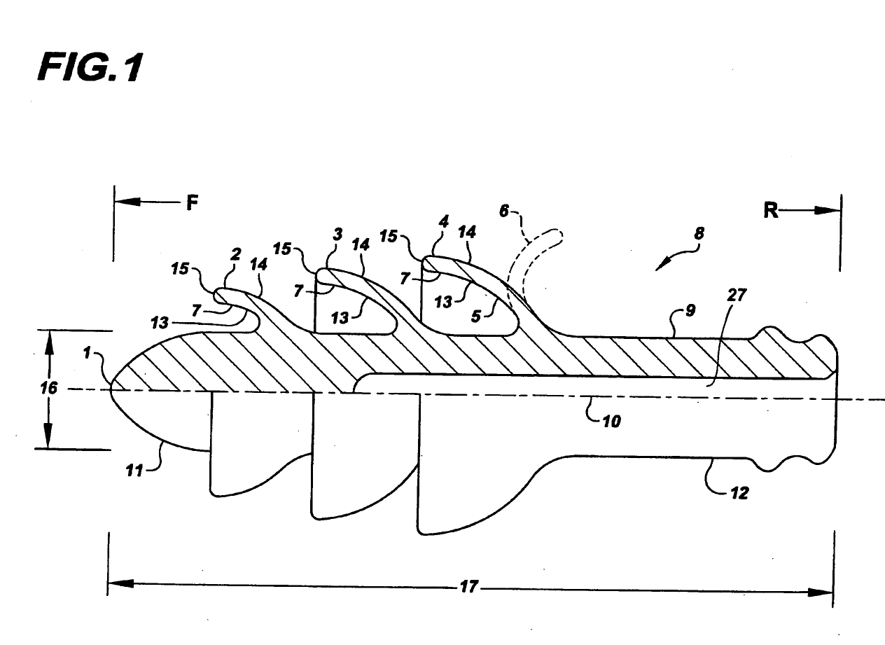

FIG. 1 shows an exemplary partial cross section half view and a partial elevation half view of the present reverse fitting ear plug 8 design. A stem 9 having a tapered or bullet shaped nose 1 extends in frontward (F) and rearward (R) directions along a stem axis 10. The stem 9 has a frontward stem portion 11 and a rearward stem portion 12. The frontward stem portion 11 precedes at least one reversible flange for creating surface contact and seal with the walls of an ear canal. An exemplary embodiment comprises a first reversible flange 2, a second reversible flange 3 and a third reversible flange 4. Other exemplary embodiments may comprise four or more reversible flanges.

The rearward portion 12 of the stem 9 serves as a handle to push the flanges (2,3,4) into the ear canal and again to pull it out. The rearward portion 12 of the stem 9 may comprise a hollowed portion 27 serving as an interface for electronic devices. Hollowed portion 27 may be lengthened or shortened for interface to specific hearing aids or electronic ear speakers for speech therapy, or other, electronic devices. The flanges (2,3,4) may be thin with radially inner surfaces 13 and outer surfaces 14 that merge and have free moving outer ends 15 that change position from generally concave frontward 6 to generally convex frontward 7 during insertion into the ear canal.

The stem 9 and flanges (2,3,4) my be integrally molded of the same material. In one embodiment, the material may comprise a memory elastomeric material. Upon easy insertion of earplug 8, the flanges (2,3,4) transform from a generally concave frontward 6 geometry to a generally convex frontward 7 geometry providing a comfortable noise blocking earplug as the material conforms to the interior wall surfaces of the ear canal in an effort to return to their original molded shape. The soft memory material allows the flanges (2,3,4) to be molded in various radially thin geometric configurations unlike earplugs of conventional design, which rely upon thickness and rigidity in the constructive material or additional stiff material to force a flange into a sealed position by applying uncomfortable pressure against the ear canal.

The approximate length 17 and diameter 16 of the earplug stem 9 are variable by design. In one exemplary embodiment, the entire length 17 of the earplug which includes the nose area 1 and the rearward stem area 12 may range from one and a quarter inch in length to one half inch in length. The diameter 16 of the exemplary embodiment may range from one half inch in diameter to one tenth of an inch in diameter. In the exemplary embodiment, the thickness of the flanges may vary from the intersection of the body of the earplug to the outer ends of the flanges. This thickness may range from ten thousands of an inch in thickness to thirty or forty thousands of an inch in thickness. In one exemplary embodiment, the geometry of the flanges (2,3,4) as they intersect the stem 9 of the earplug 8 may vary in radius dimension from ten thousands of an inch to fifty thousands of an inch, and may be radial at they extend outward to their end points. In other exemplary embodiments, the flanges (2,3,4) may be elliptical or a combination of radial, elliptical and straight lines as they are defined from the stem 9 of the earplug 8 to the outer ends of the flanges (2,3,4). Any point on inner surface 13 to another inner surface point 7 may be a variety of geometric shapes, either radial, elliptical, straight lines or any combination of these. The curve of an exemplary flange outer surface 14 may also be a variety of geometric shapes from radial, to elliptical or combinations of either. The curve of the exemplary free moving outer ends 15 may be a minimal radial curve between ten thousands of an inch in diameter and forty or fifty thousands of an inch in diameter. Again the curve of the exemplary free moving outer flange ends 15 may be a radial, elliptical, straight line or combinations of either. In the reversed convex position, the exemplary flange curve 6 may be between one eighth of an inch in diameter and half inch in diameter. The exemplary reversed flange curve 6 may be either radial, elliptical or any combination of French curves, and curve or straight lines.

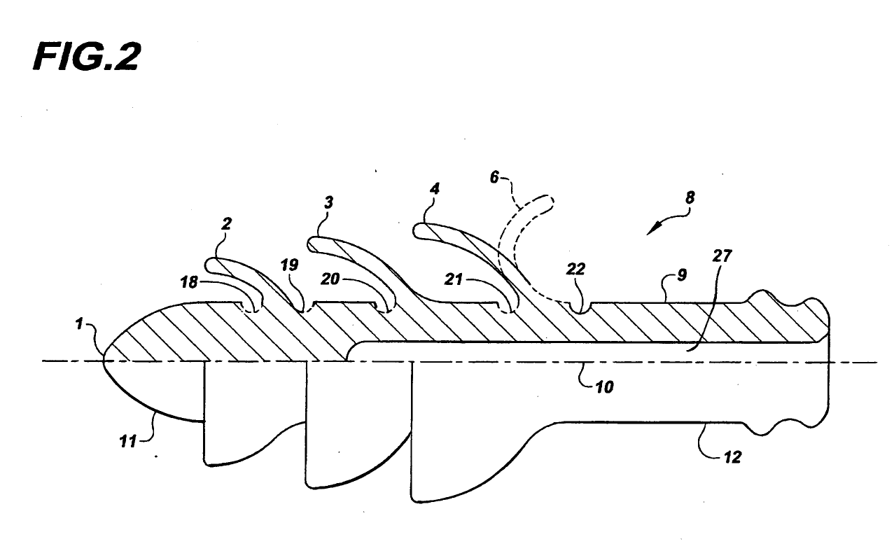

FIG. 2 shows an alternative exemplary embodiment of a partial cross section half view and a partial elevation half view. The alternative embodiment of FIG. 2 illustrates a geometric modification having radially shaped indentations (18,19,20,21,22) in the stem 9 of the earplug 8 at the base of the flanges (2,3,4). Indentations (18,19,20,21,22) create a lower pivot point for the flanges (2,3,4) in order to facilitate the hinge point of the reverse movement of the flanges, making the flanges easier to insert into the ear canal.

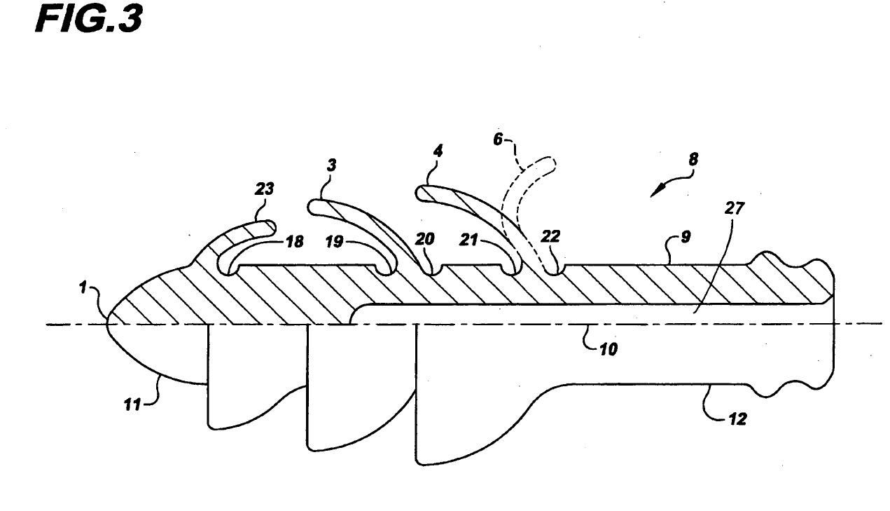

FIG. 3 shows another alternative exemplary embodiment of a partial cross section half view and a partial elevation half view of earplug 8 having a combination of conventional non-reversible convex and reverse fitting concave flanges. At least one conventional flange 23 is molded in the traditional non-reversible convex geometry of known earplug designs in combination with at least one reverse fitting concave flange. Flanges 3 and 4 are molded in the reverse fitting concave geometry, which changes direction to create a convex geometry 6 upon insertion in the ear canal. Any combination of non-reversible convex flanges 23 and reverse fitting concave flanges (3,4) may extend from the stem 9.

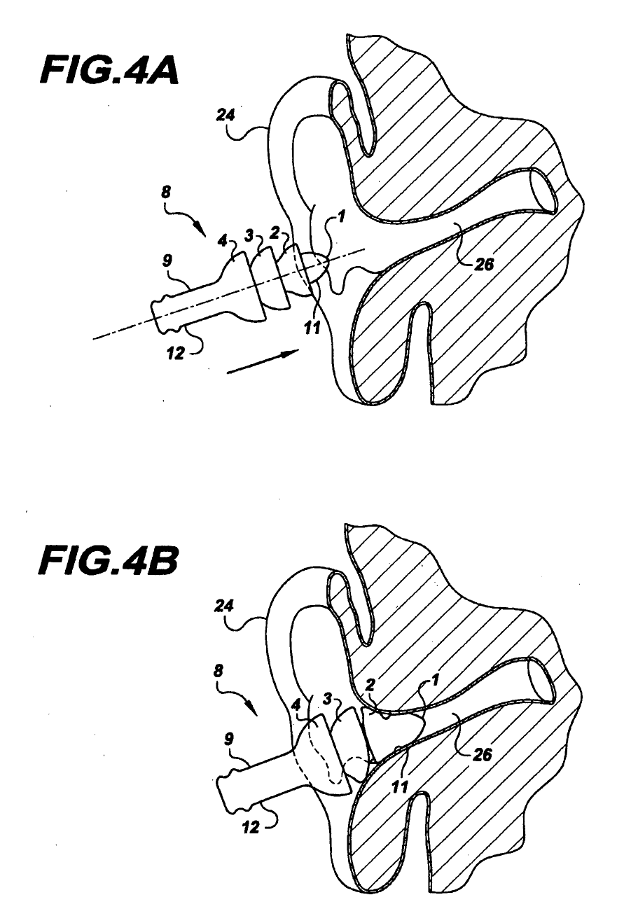

FIG. 4A illustrates an exemplary embodiment of a reverse fitting earplug 8 prior to insertion into an the ear canal 26 of an ear 24. Prior to insertion into the ear canal 26, reverse fitting flanges (2,3,4) retain their originally molded forward concave shape.

FIG. 4B illustrates an exemplary embodiment of a reverse fitting earplug 8 during insertion into an ear 24. The rearward portion 12 of the stem 9 serves as a handle to push the nose 1 and the first flange 2 into the ear canal 26. Following insertion of the earplug nose 1, the first concave flange 2 enters the ear canal 26, folding backwards in a direction reversing movement to create a convex shaped surface contact, which begins to seal the ear canal 26.

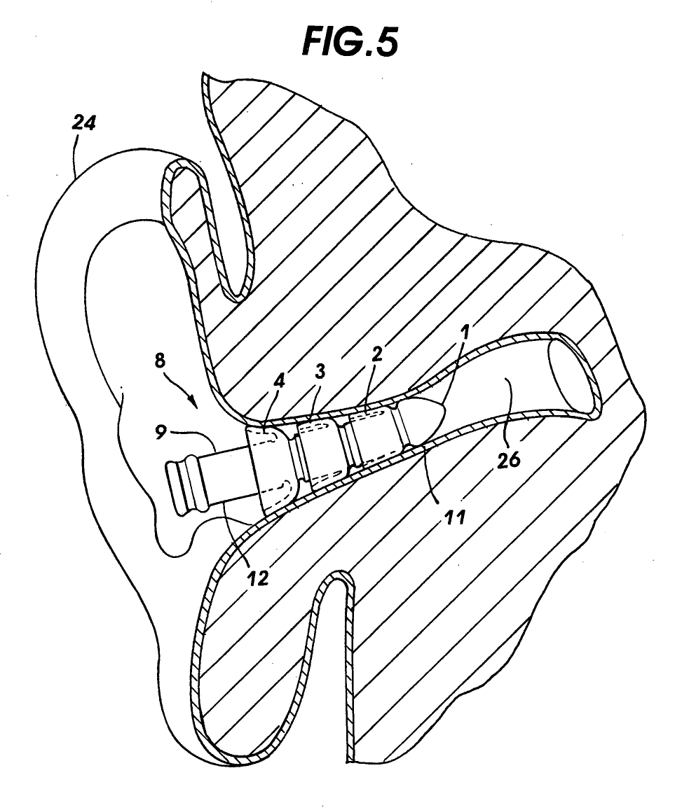

FIG. 5 illustrates an exemplary embodiment of a reverse fitting earplug after full insertion. As the user continues to push the stem 9, the second and then third concave flange (3,4) enter the ear canal 26 and reverse direction, increasing the surface contact and completing the seal. Surface contact and seal are maintained by the tendency of the flange material to return to its originally molded concave shape. Pressure forces from rigid structural material are not necessary to hold the reverse fitting earplug 8 in place. The reverse fitting earplug 8 can be comfortably worn for extended periods that cannot be achieved with conventional pressure force reliant designs. When a user removes the reverse fitting earplug 8 by pulling the stem 9, the flanges (2,3,4) automatically return to their originally molded concave shape.

In addition to superior comfort and hearing protection, the reverse fitting earplug design offers manufacturing and pricing advantages. Less severe undercuts enable a faster cycle time while the reverse fitting design requires less manufacturing material than conventional designs.

Thus, a novel and improved method and apparatus for a reverse fitting earplug have been described. The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An earplug having at least one direction changing flange.

2. The earplug of claim 1 wherein the direction changing flange reverses direction from an originally molded forward concave geometry to forward convex geometry upon insertion into an ear canal.

3. The earplug of claim 1 wherein the earplug is constructed of a plastic or elastomer.

4. The earplug of claim 1 wherein the flange geometry is hemispherical, elliptical, or any combination thereof.

5. An earplug comprising:
a stem extending in frontward and rearward directions along a stem axis; and
at least one direction reversing flange extending from the stem.

6. The earplug of claim 4 wherein the direction reversing flange is constructed of material having a memory property.

7. The earplug of claim 4 comprising three or four direction reversing flanges.

8. The earplug of claim 4 wherein the stem comprises a rearward portion serving as handle to insert and remove the earplug from an ear canal.

9. The earplug of claim 4 wherein the stem serves as in interface for electronic devices.

10. The earplug of claim 4 where in the stem and at least one flange is integrally molded of the same material.

11. The earplug of claim 4 wherein the at least one flange conforms to interior surfaces of an ear canal in an effort to return to an originally molded concave shape.

12. The earplug of claim 4 wherein the at least one flange is molded in a radially thin configuration.

13. The earplug of claim 4 wherein the stem ranges in length from one half inch to one and a quarter inches.

14. The earplug of claim 4 wherein the stem ranges in diameter from one tenth of an inch to one half inch.

15. The earplug of claim 4 wherein the thickness of the at least one flange varies from an intersection with the stem to outer ends.

16. The earplug of claim 4 wherein the thickness of the at least one flange varies from one ten thousand of an inch to forty thousands of an inch.

17. The earplug of claim 4 wherein the radial dimension of the at least one flange extends from one ten thousand of an inch to fifty thousands of an inch.

18. The earplug of claim 4 wherein outer ends of the at least one flange comprises a radial curve between one ten thousand of an inch and fifty thousands of an inch.

19. The earplug of claim 4 wherein the stem comprises radially shaped indentations at the base of the flange(s) to create a lower pivot point for the flanges(s).

20. The earplug of claim 4 further comprising at least one traditionally molded non-reversible convex flange.
